(19) United States
(12) Patent Application Publication
(10) Pub. No.: US 2017/0172927 A1
(43) Pub. Date: Jun. 22, 2017

(54) Nicotine-Containing Solid Oral Formulations and Uses Thereof

(71) Applicant: Euro-Pharm International Canada Inc., Montreal (CA)

(72) Inventors: Rolando Fusco, Montréal (CA); Leonardo Calderone, Montréal (CA)

(21) Appl. No.: 15/388,619

(22) Filed: Dec. 22, 2016

(30) Foreign Application Priority Data
Dec. 22, 2015 (CA) 2916077

(51) Int. Cl. A61K 9/28; A61K 9/20; A61K 31/465

(52) U.S. Cl. CPC A61K 9/282; A61K 31/465; A61K 9/2018; A61K 9/2059

(57) ABSTRACT

Nicotine formulations and their use in therapy, for instance, in nicotine replacement therapy are described. More specifically, the technology relates to solid oral nicotine formulations comprising nicotine, a sugar-alcohol-starch coagglomerate, a sugar alcohol, xylitol, and a coating, which may be used as lozenges for administration through the oral cavity.

### TECHNICAL FIELD

The technology generally relates to nicotine formulations and their use in therapy, for instance, in nicotine replacement therapy. More specifically, the technology relates to solid nicotine formulations, such as lozenges, for administration through the oral cavity.

### BACKGROUND

Nicotine is an alkaloid present in plants of the Solanaceae family like the tobacco plant. Apart from nicotine, tobacco leaves also include a complex mixture of compounds which, when burned and inhaled, are associated with serious health conditions, including heart diseases and lung cancer. The role of nicotine in the addiction to tobacco has now been recognized for a few decades.

However, even though the majority of users intend to stop smoking at one point or another, they are all faced with symptoms of withdrawal such as cravings, irritability, weight gain and/or depression when attempting to quit. One treatment to help reducing withdrawal symptoms includes the administration of medication, such as antidepressants or other compounds like varenicline or clonidine, all of which may present secondary effects to various degrees. For instance, the monitoring of patients using antidepressants by a medical professional on a regular basis is highly recommended to promptly identify any adverse effects.

Another approach to reduce withdrawal symptoms consists in what is called nicotine replacement therapy (NRT). In NRT, nicotine is delivered in a form which avoids risks associated with smoking, for instance, transdermally or through the mouth cavity. For instance, NRTs include nicotine patches, gums, mouth and nasal sprays, inhalers, tablets and lozenges. NRTs may also be combined together or with other smoking cessation aids such as medication or counseling.

Among NRTs, nicotine lozenges have entered the scene in more recent years. Absorption of the active is achieved through the oral mucosa. However, a burning sensation is often reported by lozenges users, which could lead to treatment interruption and relapse of the patient. A quicker absorption through the mucosa could also benefit patients in helping to reduce symptoms more spontaneously.

### SUMMARY

According to one aspect, the present application relates to a solid oral formulation (e.g. lozenges) comprising nicotine or a pharmaceutically acceptable derivative thereof, a sugar alcohol-starch coagglomerate, a sugar alcohol, xylitol, and a coating. For instance, the solid oral formulation has a total weight of less than or equal to 300 mg per dose, or of less than or equal to 250 mg per dose, or a total weight of between 150 and 250 mg per dose.

In one embodiment, the content in nicotine in the formulation ranges from about 0.2 wt % to about 5 wt %, for instance, from about 0.4 wt % to about 2.2 wt %. For instance, the nicotine amount per dose is within the range of about 0.5 mg to about 5 mg, or within the range of about 1 mg to about 4 mg. In one example the nicotine in the formulation is a resin-bound pharmaceutically acceptable nicotine derivative, for example nicotine bound to an ion-exchange resin (e.g. a polymethacrylic acid ion-exchange resin), such as nicotine polacrilex.

According to one embodiment, the sugar alcohol-starch coagglomerate comprises a sugar alcohol to starch weight ratio within the range of 90:10 to 50:50, preferably within the range of 80:20 to 65:35. In another embodiment, the content in the sugar alcohol-starch coagglomerate in the formulation is within the range of about 25 wt % to about 65 wt %, for instance about 45 wt % to 55 wt %. For instance, the sugar alcohol in the sugar alcohol-starch coagglomerate is selected from mannitol, maltitol, sorbitol, erythritol, and isomalt, preferably mannitol. In another embodiment, the starch in the sugar alcohol-starch coagglomerate is selected from corn starch or other cereal starch, potato starch, and legume starch, or a modified corn starch or other cereal starch, potato starch, or legume starch.

In a further embodiment, the sugar alcohol (other than the sugar alcohol present in the coagglomerate, i.e. a free sugar alcohol) is selected from mannitol, maltitol, sorbitol, erythritol, and isomalt, preferably mannitol. For instance the content in free sugar alcohol, i.e. excluding the sugar alcohol from the sugar alcohol-starch coagglomerate, in the formulation is in the range of about 10 wt % to about 35 wt %, or about 18 wt % to about 26 wt %.

In yet another embodiment, the xylitol content in the formulation is in the range of about 5 wt % to about 25 wt %, or in the range of about 8 wt % to about 15 wt %, or of about 12 wt %.

In a further embodiment, the coating of the solid oral formulation comprises a starch or modified starch or polymer coating, a sweetener and a flavoring agent. For instance, the starch or modified starch is a legume starch or modified starch, e.g. pea starch or modified pea starch. For example, the coating polymer comprises a polyvinyl alcohol, a partially hydrolyzed polyvinyl alcohol, and/or a polyethylene glycol.

For example, the sweetener in the coating is sorbitol and/or monk fruit (Momordica). According to one example, the flavoring agent in the coating comprises mint, peach, grape, coffee, or orange flavor, or a combination thereof. In one embodiment, the flavoring agent comprises citric acid. The coating may comprise further excipients such as pigments (e.g. titanium oxide) or lubricants such as stearic acid or talc.

The application also further relates to the use of a formulation as herein defined for treating, reducing, and/or alleviating at least one tobacco withdrawal symptom, and to methods of treating, reducing, and/or alleviating at least one tobacco withdrawal symptom in a subject in need thereof, comprising administering an effective amount of a solid oral formulation as herein defined. The formulation as herein defined in a form suited for administration to the mouth cavity, for instance, in the form of lozenges.

### DETAILED DESCRIPTION

All technical and scientific terms used herein have the same meaning as commonly understood by one ordinary skilled in the art to which the present technology pertains. For convenience, the meaning of certain terms and phrases used herein are provided below.

To the extent the definitions of terms in the publications, patents, and patent applications incorporated herein by reference are contrary to the definitions set forth in this specification, the definitions in this specification control. The section headings used herein are for organizational purposes only, and are not to be construed as limiting the subject matter disclosed.

The terminology used herein is for the purpose of describing particular embodiments only and is not intended to be limiting. It should be noted that, the singular forms “a”, “an”, and “the” include plural forms as well, unless the content clearly dictates otherwise. Thus, for example, reference to a composition containing “a coating polymer” also contemplates a mixture of two or more coating polymers. It should also be noted that the term “or” is generally employed in its sense including “and/or” unless the content clearly dictates otherwise. Furthermore, to the extent that the terms “containing”, “contains”, “including”, “includes”, “having”, “has”, “with”, or variants thereof are used in either the detailed description and/or the claims, such terms are intended to be inclusive in a manner similar to the term “comprising”.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of ±20%, or ±10% of a given value. Where particular values are described in the application and claims, unless otherwise stated the term “about” meaning within an acceptable error range for the particular value should be assumed.

The expression “solid oral formulation” in the present application refers to an orally dissolvable, disintegrable or dispersible composition for transmucosal absorption of an active ingredient. Examples of solid oral formulations include, without limitation, lozenges, tablets, sticks, canes, troches, and the like. A preferred formulation includes lozenges which may be in any shape, for instance, round, diamond, rectangular, octagonal, oval, spherical, cylindrical, flattened disk, biconvex, etc.

The term “nicotine” refers both to the free base of nicotine as well as to pharmaceutically acceptable salts or derivatives of nicotine. Pharmaceutically acceptable salts refer to salts of nicotine which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66: 1-19 (1977), incorporated herein by reference in its entirety and for all purposes. A “pharmaceutically acceptable derivative” includes any non-toxic prodrug, conjugate, complex, or other derivative of nicotine that, upon administration to a subject, is capable of providing nicotine, either directly or indirectly. Examples of derivatives include nicotine bound to an ion exchange resin (e.g. nicotine polacrilex and the like).

The expression “sugar alcohol-starch coagglomerate” refers to a homogeneous mixture of a solid sugar alcohol and a starch, for instance obtained by atomisation. Examples of orodispersible mannitol-starch coagglomerates are described in PCT published application No WO2010/001063, which is incorporated herein by reference in its entirety for all purposes.

The expression “sugar alcohol” designates an alcohol which is a reduced form of the aldehyde group present in its corresponding sugar molecule. Examples of sugar alcohols include, without limitation, mannitol, maltitol, sorbitol, erythritol, isomalt, and combinations thereof.

The expression “flavoring agents” as used herein, designates natural or synthetic agents which provide an appealing taste to the formulation, masks the taste of nicotine and/or excipients, and/or help in reducing the burning sensation associated with nicotine formulations when administered through the mouth cavity. Examples of flavoring agents include, without limitations, mint, spearmint, pepper mint, wintergreen, menthol, eucalyptus, cinnamon, cocoa, vanilla, aniseeds, liquorice, fruit flavor (e.g. grape, cherry, strawberry, raspberry, melon, banana, papaya, mango, peach, pineapple, cranberry, apple, orange, lemon, lime, etc.), citric acid, coffee, tea, tobacco flavor, artificial or natural brew and liquor flavors, and the like, or a combination thereof.

The expression “excipient” refers to inert carriers or ingredients which may be included in the formulation to obtain the desired flowability, compressability, stability, or aesthetic. For instance, excipients include fillers, binders, pigments, preservatives, buffering agents, glidants, lubricants, colorants, etc. Such excipients may be included in the formulation's core or in the coating thereof.

The solid oral formulations as herein defined preferably include nicotine in any form, i.e. nicotine or a pharmaceutically acceptable derivative thereof, a sugar alcohol-starch coagglomerate, a sugar alcohol, xylitol, and a coating. For instance, the solid oral formulation has a total weight of less than or equal to 300 mg per dose, or of less than or equal to 250 mg per dose, or a total weight of between 150 and 250 mg per dose.

Nicotine is preferably present in the formulation in an amount which ranges from about 0.2 wt % to about 5 wt %, for instance, from about 0.4 wt % to about 2.2 wt %. For example, nicotine is present in the formulation at a concentration which is adapted to deliver a dose of about 0.5 mg to about 5 mg, or about 1 mg to about 4 mg, for instance, a dose of about 1 mg, 2 mg, 3 mg, or 4 mg of nicotine in one solid oral dose (e.g. in one lozenge).

The nicotine may be included in the formulation as a free base or as an acceptable salt. In the alternative, the nicotine present in the formulation may be within an inclusion complex, such as a resin-bound complex, for example nicotine bound to an ion-exchange resin (e.g. a polymethacrylic acid ion-exchange resin). The resin-bound nicotine complex may have a content of about 10 wt % to about 40 wt % of nicotine, for instance, around 20 wt %. The content in resin-bound nicotine complex in the formulation may be within the range of about 1 wt % to about 25 wt %, or about 2 wt % to about 10 wt %. The amount in resin-bound nicotine complex in the formulation may be within the range of about 2.5 mg to about 50 mg per dose, or about 5 mg to about 20 mg per dose.

The formulation further includes a sugar alcohol-starch coagglomerate which comprises a sugar alcohol/starch weight ratio within the range of 90:10 to 50:50, preferably within the range of 80:20 to 65:35. The content in the sugar alcohol-starch coagglomerate in the formulation may be within the range of about 25 wt % to about 65 wt %, for instance about 45 wt % to 55 wt %. The content in the sugar alcohol-starch coagglomerate in one dose of the formulation may be within the range of about 80 mg to about 120 mg, for instance, about 100 mg.

The sugar alcohol-starch coagglomerate essentially consists in an atomized, homogeneous mixture of a sugar alcohol and a starch. For instance, the sugar alcohol in the sugar alcohol-starch coagglomerate is selected from mannitol, maltitol, sorbitol, erythritol, and isomalt, preferably mannitol. The starch in the sugar alcohol-starch coagglomerate may be selected from corn starch or other cereal starch, potato starch, and legume starch, or a modified corn starch or other cereal starch, potato starch, or legume starch. In a preferred example, the sugar alcohol-starch coagglomerate is Pearlitol™ Flash.

The formulation also includes a further sugar alcohol in addition to the sugar alcohol present in the coagglomerate (i.e. a free sugar alcohol). For example, the free sugar alcohol is selected from mannitol, maltitol, sorbitol, erythritol, and isomalt, preferably mannitol, for instance Pearlitol™ 200SD. For instance, the content in free sugar alcohol, i.e. excluding the sugar alcohol from the sugar alcohol-starch coagglomerate, in the formulation is in the range of about 10 wt % to about 35 wt %, or about 18 wt % to about 26 wt %. The free sugar alcohol content in one dose of the formulation may be within the range of about 25 to about 70 mg, or about 30 mg to about 55 mg.

The formulation of the present application also further comprises xylitol, the xylitol content in the formulation being in the range of about 5 wt % to about 25 wt %, or in the range of about 8 wt % to about 15 wt %, or of about 12 wt %. For instance, the formulation comprises, in one dose, about 15 mg to about 35 mg of xylitol, preferably, about 20 mg to about 30 mg of xylitol. In one example, the xylitol used is of the brand Xylisorb™ 200DC, from Roquettes Freres. Xylitol is included in the formulation to enhance absorption of nicotine.

While the formulation may comprise further excipients within its core, the total content in nicotine or a pharmaceutically acceptable derivative thereof, sugar alcohol-starch coagglomerate, sugar alcohol, and xylitol accounts for at least 75% of the total weight of a dose of the formulation, for instance, at least 80%. When excluding the coating, the total amount of these elements accounts for at least 80%, or at least 85%, or about 90% of the total weight of a dose of the formulation. Additional excipients are as herein defined and may include, for instance, microcrystalline cellulose, and/or a lubricant such as magnesium stearate. In any one of the present embodiments, the formulation may be substantially free of water, or of any buffer.

The formulation further comprises a coating which covers the surface of the solid oral formulation's core. For instance, the coating of the solid oral formulation comprises a starch or modified starch, a sweetener and a flavoring agent. For instance, the starch or modified starch is a legume starch or modified starch, e.g. pea starch or modified pea starch. In another example, the coating of the solid oral formulation comprises at least one coating polymer, a sweetener and a flavoring agent. For instance the coating polymer comprises polyvinyl alcohol, partially hydrolyzed polyvinyl alcohol, and/or polyethylene glycol. For example, the coating includes the ReadiLycoat™ (Roquettes Freres) or an Opadry™ coating (Colorcon), a flavoring agent and a sweetener.

The sweetener in the coating is a natural or artificial sweetener. For example, the sweetener in the coating may be a sugar alcohol like sorbitol, and/or a fruit extract like monk fruit (Momordica). According to one example, the flavoring agent in the coating is selected from mint, peach, grape, coffee, orange, or a combination thereof. Preferably, the flavoring agent comprises citric acid, which helps reducing the burning sensation associated with nicotine. The coating may comprise further excipients such as pigments (e.g. titanium oxide) and/or lubricants such as stearic acid or talc.

An “effective amount” of the formulation as herein described includes an amount of nicotine that will elicit the biological or medical response in a subject that is being sought. For example, an effective amount is defined as an amount which would, as compared to a corresponding subject who has not received such amount, result in treatment, prevention, reduction or alleviation of at least one symptom of nicotine withdrawal. Nicotine withdrawal symptoms include cravings (urges to use tobacco), anxiety, irritability, depression, and weight gain. For instance, an amount per dose (i.e. delivered in 1 lozenge) may range from about 1 mg to about 4 mg.

As used herein, the terms “treatment,” “treat,” and “treating” refer to alleviating, delaying the onset of, or reducing at least one symptom associated with nicotine withdrawal, as described herein. In some embodiments, treatment may be administered after one or more symptoms have developed. In other embodiments, treatment may be administered to a susceptible individual prior to the onset of symptoms. For instance, the product is used at the moment a nicotine craving develops and this nicotine craving needs to be suppressed. That way, instead of using a tobacco product, the individual being treated may take a dose of the formulation to remove, alleviate or delay the immediate requirement for nicotine.

The term “subject” as used herein refers to a subject susceptible of encountering nicotine withdrawal symptoms, for instance, a human subject who has been exposed to nicotine for a period of time, e.g. a human who has been smoking tobacco products, for instance, for at least 2 months, or at least 6 months, or at least 1 year, and for whom exposition is stopped or substantially reduced (e.g. a subject quitting smoking).

It should also be understood that the dose, daily dosage and/or frequency of administration for any particular subject will depend upon a variety of factors, including age, body weight, general health, time of administration, drug combination, the frequency and duration of previous exposure to nicotine (e.g. smoking habits), the judgment of the treating physician, and the frequency and severity of nicotine withdrawal symptoms. For instance, one or two dose(s) may be administered on an “as needed” basis or as determined by a medical professional. The total daily usage may not exceed the amount recommended by local health authorities. The present formulation may also be used in combination or coincidental with other NRT(s) (e.g. patches, sprays, gums), tobacco cessation drugs, and/or behavioral counseling, given that combination of the formulation with other NRT(s) is allowed and accepted by health authorities and that the total daily dose of nicotine absorbed by the subject is at or below the limit accepted by such authorities.

The Example set forth herein below provide an example of production of a formulation as defined herein. Unless otherwise indicated, all numbers expressing quantities of ingredients, conditions, concentrations, and so forth used in the specification and claims are to be understood as being modified in all instances by the term “about.” At the very least, each numerical parameter should at least be construed in light of the number of significant digits and by applying ordinary rounding techniques. Accordingly, unless indicated to the contrary, the numerical parameters set forth in the present specification and attached claims are approximations that may vary depending upon the properties sought to be obtained. Notwithstanding that the numerical ranges and parameters setting forth the broad scope of the embodiments are approximations, the numerical values set forth in the specific examples are reported as precisely as possible. Any numerical value, however, inherently contains certain errors resulting from variations in experiments, testing measurements, statistical analyses and so on.

The following is to be construed as merely illustrative, and not limitations of the preceding disclosure in any way whatsoever. Those skilled in the art will promptly recognize appropriate variations from the procedures both as to ingredients and as to conditions and techniques used. Ingredients used are, for instance, generally available USP grade commercial materials.

### EXAMPLE

Exemplary formulation ingredients are summarized in Table 1 along with the quantity of each ingredient expressed as the amount per unit dose (per lozenge).

TABLE 1

| | Amount | | Amount | | Amount | | Amount | |
|---|---|---|---|---|---|---|---|---|
| Material | (1 mg/dose) | | (2 mg/dose) | | (1 mg/dose) | | (2 mg/dose) | |
| Nicotine | 5 | mg | 10 | mg | 15 | mg | 20 | mg |
| Polacrilex | | | | | | | | |
| Mannitol-Starch | 100 | mg | 100 | mg | 100 | mg | 100 | mg |
| Coagglomerate* | | | | | | | | |
| Mannitol* | 51 | mg | 46 | mg | 41 | mg | 36 | mg |
| Microcrystalline | 16 | mg | 16 | mg | 16 | mg | 16 | mg |
| cellulose | | | | | | | | |
| Xylitol* | 25 | mg | 25 | mg | 25 | mg | 25 | mg |
| Magnesium | 3 | mg | 3 | mg | 3 | mg | 3 | mg |
| stearate | | | | | | | | |
| Coating** | 10-16 | mg | 10-16 | mg | 10-16 | mg | 10-16 | mg |
| Total | 216 | mg | 216 | mg | 216 | mg | 216 | mg |

*From Roquette Fr [symbol] res
**Coating comprises ReadiLycoat ™ (Roquette Fr [symbol] res) or Opadry ™ (Colorcon), a flavoring agent comprising citric acid (between 3 mg and 6 mg of flavoring agent)

A formulation is prepared by mixing about 90% of the microcrystalline cellulose NF and Nicotine Polacrilex in an appropriate blender to afford mixture A. Mixture A and xylitol are combined and blended to obtain premix B.

The rest of microcrystalline cellulose NF (about 10%) is combined with magnesium stearate and mixed to obtain premix C. In a suitable blender, the mannitol-Starch coagglomerate (e.g. Pearlitol™ Flash) and Mannitol (e.g. Pearlitol™ 200SD) are combined to afford blend D.

To blend D, is added premix B and the ingredients are mixed to afford blend E. Premix C is added to blend E and the composition further mixed.

The preceding steps may each independently further include passing the premix, blend or mixture though a, for example, 20 or 30 mesh screen.

The composition is then molded and compressed to obtain lozenges in the form of oval tablets. These oval tablets are then film coated with a suspension comprising the coating, the flavoring agent and the sweetener.

The core of each of the above formulations obtained disintegrates in about 1 minute in the mouth. Xylitol is included in the composition to improve absorption of nicotine. Citric acid is included in the coating's flavoring agent to help reducing the burning sensation caused by nicotine.

Although the invention has been illustrated and described with respect to one or more implementations, equivalent alterations and modifications will occur to others skilled in the art upon the reading and understanding of this specification. In addition, while a particular feature of the invention may have been disclosed with respect to only one of several implementations, such feature may be combined with one or more other features of the other implementations as may be desired and advantageous for any given or particular application.

Accordingly, it is understood that the examples and embodiments described herein are for illustrative purposes only and that various modifications or changes in light thereof will be suggested to persons skilled in the art and are to be included within the spirit and purview of this application and scope of the appended claims. Any publication, document, patent, patent application or publication referred to herein should be construed as incorporated by reference each in their entirety for all purposes.

1. A solid oral formulation comprising nicotine or a pharmaceutically acceptable derivative thereof, a sugar alcohol-starch coagglomerate, a sugar alcohol, xylitol, and a coating.

2. The solid oral formulation of claim 1, wherein the solid oral formulation has a total weight of less than or equal to 300 mg per dose, less than or equal to 250 mg per dose, or between 150 and 250 mg per dose.

3. The solid oral formulation of claim 1, wherein the content in nicotine in the formulation ranges from about 0.2 wt % to about 5 wt %, or from about 0.4 wt % to about 2.2 wt %.

4. The solid oral formulation of claim 1, wherein the nicotine amount per dose is within the range of about 1 mg to about 4 mg.

5. The solid oral formulation of claim 1, wherein said nicotine is in the form of a resin-bound pharmaceutically acceptable nicotine derivative.

6. The solid oral formulation of claim 5, wherein said resin-bound pharmaceutically acceptable nicotine derivative is nicotine bound to an ion-exchange resin.

7. The solid oral formulation of claim 1, wherein said sugar alcohol-starch coagglomerate comprises a sugar alcohol to starch weight ratio within the range of 90:10 to 50:50, preferably within the range of 80:20 to 65:35.

8. The solid oral formulation of claim 1, wherein the content in sugar alcohol-starch coagglomerate in the formulation is within the range of about 25 wt % to about 65 wt %, for instance about 45 wt % to 55 wt %.

9. The solid oral formulation of claim 1, wherein the sugar alcohol in the sugar alcohol-starch coagglomerate is selected from mannitol, maltitol, sorbitol, erythritol, and isomalt, e.g. mannitol.

10. The solid oral formulation of claim 1, wherein the starch in the sugar alcohol-starch coagglomerate is selected from corn starch or other cereal starch, potato starch, and legume starch, or a modified corn starch or other cereal starch, potato starch, or legume starch.

11. The solid oral formulation of claim 1, wherein the sugar alcohol is selected from mannitol, maltitol, sorbitol, erythritol, and isomalt, preferably mannitol.

12. The solid oral formulation of claim 1, wherein the content in sugar alcohol, excluding the sugar alcohol from the sugar alcohol-starch coagglomerate, in the formulation is in the range of about 10 wt % to about 35 wt %, or about 18 wt % to about 26 wt %.

13. The solid oral formulation of claim 1, wherein the xylitol content in the formulation is in the range of about 5 wt % to about 25 wt %, about 8 wt % to about 15 wt %, or about 12 wt %.

14. The solid oral formulation of claim 1, wherein said coating comprises a starch or modified starch or a coating polymer, a sweetener and a flavoring agent.

15. The solid oral formulation of claim 14, wherein the starch or modified starch is a legume starch or modified legume starch or a pea starch or modified pea starch.

16. The solid oral formulation of claim 14, wherein the coating polymer is polyvinyl alcohol, partially hydrolyzed polyvinyl alcohol, and/or polyethylene glycol.

17. The solid oral formulation of claim 14, wherein the sweetener is sorbitol and/or a fruit extract (e.g. a monk fruit extract, i.e. Momordica).

18. The solid oral formulation of claim 14, wherein the flavoring agent comprises citric acid.

19. The solid oral formulation of claim 14, wherein the flavoring agent comprises mint, peach, grape, coffee, or orange flavor, or a combination thereof.

20. The solid oral formulation of claim 14, wherein the coating further comprises a pigment (e.g. titanium oxide) and/or a lubricant (e.g. stearic acid or talc).

21. Method for treating, reducing, or alleviating at least one tobacco withdrawal symptom (e.g. nicotine craving) in a subject in need thereof, comprising administering to said subject an effective amount of a solid oral formulation as defined in claim 1 to the mouth cavity.
